(19) United States
(12) Patent Application Publication
(10) Pub. No.: US 2021/0077515 A1
(43) Pub. Date: Mar. 18, 2021

(54) COMPOSITIONS CONTAINING BETA-NICOTINAMIDE MONONUCLEOTIDE AND CORDYCEPS SINENSIS AND USE THEREOF

(71) Applicant: Hangzhou Huadong Medicine Comprehensive Health Development Co., Ltd., Hangzhou (CN)

(72) Inventors: Bangliang LI, Hangzhou (CN); Caihong XU, Hangzhou (CN)

(21) Appl. No.: 17/105,563

(22) Filed: Nov. 26, 2020

(30) Foreign Application Priority Data
Oct. 12, 2020 (CN) 202011082960.9

(51) Int. Cl. A61K 31/706; A61K 36/068; A61K 47/38; A61K 47/12

(52) U.S. Cl. CPC A61K 31/706; A61K 36/068; A61K 9/48; A61K 47/12; A61K 47/38

(57) ABSTRACT

The invention discloses a composition containing β-nicotinamide mononucleotide and Cordyceps sinensis. The ratio of β-nicotinamide mononucleotide to Cordyceps sinensis can be 2-5:1. The composition has the effects of improving NAD+ level in vivo, repairing DNA, maintaining cell health, promoting NADP synthesis, and increasing immunity. The combination of β-nicotinamide mononucleotide and Cordyceps sinensis can strengthen various effects, create synergistic effect, and thus the present composition is suitable for the majority of people.

### FIELD OF THE INVENTION

The invention relates to the technical field of biological medicine and health food, in particular to a composition containing β-nicotinamide mononucleotide and Cordyceps sinensis and use thereof.

### BACKGROUND OF THE INVENTION

β-nicotinamide mononucleotide (NMN) is the product of the reaction of nicotinamide phosphate ribose transferase (Nampt) with nicotinamide and the like. It is also the key precursor of nicotinamide adenine dinucleotide (NAD+) salvage synthesis pathway in mammals.

In mammals, NMN is generated by nicotinamide (NAM) under the catalysis of Nampt, and then NMN is catalyzed by nicotinamide mononucleotide adenosine transferase (Nmnat) to produce NAD+. In other words, NMN plays its physiological functions by transforming into NAD+ in vivo, such as activating NAD+ substrate dependent enzyme Sirt1 (histone deacetylase, also known as silent regulatory protein), regulating cell survival and death, maintaining redox status, etc.

It has been found that by regulating the level of NMN in organism, MIN has good therapeutic and repairing effects on cardiovascular and cerebrovascular diseases, neurodegenerative diseases and aging degenerative diseases. In addition, NMN can also protect and repair islets function, increase insulin secretion, prevent and treat metabolic diseases such as diabetes and obesity by participating in and regulating the endocrine of the body. Therefore, NMN has a wide range of applications in medical treatment and functional food.

Cordyceps sinensis is a kind of rare Chinese medicinal material. In addition to sugar, fat, crude fiber and other general components, it also contains mannitol, ergosterol, adenosine, cordyceptic acid, 3-deoxyadenosine, vitamin B12 and other active components. Its nourishing and therapeutic functions are above ginseng and pilose antler. Natural Cordyceps sinensis has a long growth period and is distributed in high altitude meadow area. It is scarce, difficult to collect and very expensive.

According to the literature reports, Cordyceps sinensis has a variety of effects, such as tonifying the kidney and strengthening Yang, tonifying the lung and relieving asthma, stopping bleeding and resolving phlegm, etc., and also has the effect of improving human immunity. However, it has not been reported that it can enhance the level of NAD+ in the body, repair DNA, maintain cell health, or promote the synthesis of NADP.

### SUMMARY OF INVENTION

For solving the technical problems in the prior art, the present invention provides a composition suitable for improving the level of NAD+ in vivo, repairing DNA, maintaining cell health and promoting NADP synthesis by the combination of β-nicotinamide mononucleotide and Cordyceps sinensis.

The present invention discloses a composition containing β-nicotinamide mononucleotide and. Cordyceps sinensis.

Preferably, the Cordyceps sinensis suitable for the present invention is an artificially fermented mycelium of Cordyceps sinensis.

According to some embodiments in the present invention, the weight ratio of β-nicotinamide mononucleotide to Cordyceps sinensis in the present composition is 2-5:1, and preferably 3:1.

According to some embodiments in the present invention, the composition of the invention comprises:

2-5 parts by weight of β-nicotinamide mononucleotide;

1 part by weight of Cordyceps sinensis;

1.5-3 parts by weight of microcrystalline cellulose; and

0.03-0.12 part by weight of magnesium stearate.

According to some more preferred embodiments in the present invention, the composition of the invention comprises:

3 parts by weight of β-nicotinamide mononucleotide;

1 part by weight of Cordyceps sinensis;

2 parts by weight of microcrystalline cellulose; and

0.03 part by weight of magnesium stearate.

The composition of the present invention can be made into a solid preparation. Preferably, the solid preparation of the composition is a capsule.

The present invention also provides the use of the composition in improving NAD+ level in vivo, repairing DNA, maintaining cell health and promoting NADP synthesis.

### DETAILED DESCRIPTION OF THE INVENTION

The Cordyceps sinensis suitable for the present invention includes artificially fermented mycelium of Cordyceps sinensis and natural Cordyceps sinensis. From the perspective of environmental protection, cost reduction and industrialization, artificially fermented mycelium of Cordyceps sinensis is more suitable for the present invention than natural Cordyceps sinensis. Artificially fermented mycelium of Cordyceps sinensis is a Cordyceps sinensis mycelium obtained by industrial fermentation of Hirsutella sinensis which is an anamorph of Cordyceps sinensis (Berkeley) Sacc. At present, a number of manufacturers can produce Cordyceps sinensis mycelium by artificial fermentation.

The weight ratio of β-nicotinamide mononucleotide and Cordyceps sinensis used in the composition of the present invention can be 2-5:1, preferably 3:1.

The invention discloses a composition containing β-nicotinamide mononucleotide and Cordyceps sinensis, comprising:

2-5 parts by weight of β-nicotinamide mononucleotide;

1 part by weight of Cordyceps sinensis;

1.5-3 parts by weight of microcrystalline cellulose; and

0.03-0.12 part by weight of magnesium stearate.

According to some more preferred embodiments in the present invention, the composition of the invention comprises:

3 parts by weight of β-nicotinamide mononucleotide;

1 part by weight of Cordyceps sinensis;

2 parts by weight of microcrystalline cellulose; and

0.03 part by weight of magnesium stearate.

The Cordyceps sinensis is preferably the artificially Fermented mycelium of Cordyceps sinensis, and the microcrystalline cellulose is preferably microcrystalline cellulose PH102.

The composition of the invention can be made into solid preparations, such as capsules, tablets, etc. Depending on the specific preparation, flavoring agent, colorant, coating (film coating, sugar coating) and the like can be added into the composition.

The composition disclosed in the invention is preferably made into capsules.

The solid preparation of the composition of the invention can be prepared by conventional methods.

The composition of the invention has the effects of improving a subject's (such as a human) NAD+ level in vivo, repairing the subject's DNA, maintaining the subject's cell health, promoting the subject's NADP synthesis, and also has the effect of increasing the subject's immunity.

The combination of β-nicotinamide mononucleotide and Cordyceps sinensis in the invention can have improved efficacies than either component alone. Moreover, the combination has synergistic effect which is stronger than the sum of the effects of either β-nicotinamide mononucleoside or Cordyceps sinensis alone. Therefore, the combination is suitable for the general population.

### EXAMPLES

### Example 1

| Raw and Auxiliary materials | Amount (g) |
|---|---|
| β-nicotinamide mononucleotide | 100 |
| Cordyceps sinensis mycelium powder | 50 |
| microcrystalline cellulose PH102 | 75 |
| magnesium stearate | 1.5 |

The materials were mixed evenly and pressed to prepare tablets (1,000 tablets were obtained).

### Example 2

| Raw and Auxiliary Materials | Amount (g) |
|---|---|
| β-nicotinamide mononucleotide | 1,500 |
| Cordyceps sinensis mycelium powder | 500 |
| microcrystalline cellulose PH102 | 1,000 |
| magnesium stearate | 15 |

The materials were mixed evenly and filled into No. 1 gelatin hollow capsules to thereby obtain 10,000 capsules.

### Example 3

| Raw and Auxiliary Materials | Amount (g) |
|---|---|
| β-nicotinamide mononucleotide | 25,000 |
| Cordyceps sinensis mycelium powder | 5,000 |
| microcrystalline cellulose PH102 | 15,000 |
| magnesium stearate | 600 |

The materials were mixed evenly and filled into No. 0 gelatin hollow capsules to thereby obtain 100,000 capsules.

What is claimed is:

1. A composition comprising:
β-nicotinamide mononucleotide and Cordyceps sinensis.

2. The composition according to claim 1, wherein the Cordyceps sinensis is an artificially fermented mycelium of Cordyceps sinensis.

3. The composition according to claim 1, wherein the weight ratio of β-nicotinamide mononucleotide to Cordyceps sinensis is 2-5:1.

4. The composition according to claim 3, wherein the weight ratio of β-nicotinamide mononucleotide and Cordyceps sinensis is 3:1.

5. The composition according to claim 1, comprising:
2-5 parts by weight of β-nicotinamide mononucleotide;
1 part by weight of Cordyceps sinensis;
1.5-3 parts by weight of microcrystalline cellulose; and
0.03-0.12 parts by weight of magnesium stearate.

6. The composition according to claim 5, comprising:
3 parts by weight of β-nicotinamide mononucleotide;
1 part by weight of Cordyceps sinensis;
2 parts by weight of microcrystalline cellulose; and
0.03 part by weight of magnesium stearate.

7. The composition according to claim 5, wherein the Cordyceps sinensis is an artificially fermented mycelium.

8. The composition according to claim 5, which is in the form of a solid preparation.

9. The composition according to claim 5, wherein the solid preparation is a capsule.

10. A method of improving NAD+ level of a subject, repairing DNA of a subject, maintaining cell health of a subject, or promoting NADP synthesis of a subject, using the composition according to claim 1.
